(19) United States
(12) Patent Application Publication
(10) Pub. No.: US 2008/0009003 A1
(43) Pub. Date: Jan. 10, 2008

(54) SINGLE PROMOTER INHIBITOR

(75) Inventor: Alan M. Tartakoff, Cleveland Heights, OH (US)

Correspondence Address: TAROLLI, SUNDHEIM, COVELL& TUMMINO L.L.P., SUITE 1700, 1300 EAST NINTH STREET, CLEVELAND, OH, 44114, US

(73) Assignee: Case Western Reserve University

(21) Appl. No.: 11/675,394

(22) Filed: Feb. 15, 2007

Related U.S. Application Data
(60) Provisional application No. 60/773,442, filed on Feb. 15, 2006.

(51) Int. Cl. C12Q 1/68

(52) U.S. Cl. 435/6

(57) ABSTRACT

A method of identifying complementary DNA (cDNA) that contributes to cell phenotype includes providing a plurality of cells. The cells are effected so that each cell expresses a single ectopic cDNA. The cells are propagated for a growth interval in an environment that allows the cells to compete against one another. The relative abundance of cDNAs from the propagated cells is determined.

### RELATED APPLICATION

The present application claims priority from U.S. Provisional Application No. 60/773,442, filed Feb. 15, 2006, which is herein incorporated by reference in its entirety.

At least a portion of the subject matter described in the present application was supported in part by NSF Grant No. MCB-0104523. Accordingly, the U.S. government may have certain rights in this invention.

### FIELD OF THE INVENTION

The present invention relates to genetic engineering and screening methods useful in the identification of gene targets and to methods and compositions for analysis of genetic and protein interactions.

### BACKGROUND OF THE INVENTION

Traditional yeast synthetic lethal screens use a “plasmid shuffling” strategy. The first step involves constructing a strain where the target gene has been deleted. This gene is then re-introduced into the cell with a low-copy plasmid that also contains a marker (e.g., ADE2) that can be used to select for the plasmid or detect the loss of the plasmid. The strain is then mutagenized and allowed to grow in the absence of selection for the plasmid. Under this condition, the majority of strains will lose the plasmid over time, which results in colonies with a sectored appearance. For example, yeast strains missing the ADE2 generate red colonies due to the accumulation of an intermediate. However, a cell that has acquired a mutation in a gene that is synthetically lethal with the target gene will generate a colony that is completely white. The colonies are white because any cells that lose the plasmid die due to the lethal nature of the double mutant.

Another approach for performing synthetic lethal screens in yeast involves generating a conditional lethal mutant for a target gene. The strain bearing such a mutation is then mutagenized and screened for second-site mutations that specifically exacerbate its temperature sensitivity. This approach has successively been used to identify proteins involved in the translocation step of protein secretion.

### SUMMARY OF THE INVENTION

The present invention relates to a method of identifying complementary DNA (cDNA)(and/or genes) that contribute to cell phenotype, such as cDNA/genes that promote or inhibit cell growth. In the method, a plurality of cells are provided. The plurality of cells can include, for example, yeast cells or eukaryotic cells (e.g., animal cells). Each cell can be effected to express a single ectopic cDNA by, for example, transfecting (or infecting) the cells with an ectopic cDNA library vector (e.g., viral and/or plasmid) at a low multiplicity. The effected cells can be propagated (or grown) for a growth interval (e.g., more than one generation) in an environment that allows the cells to compete against one another. This environment can be, for example, a culture or an animal (e.g., immunodeficient mouse). After the growth interval, which allows the infected (or transfected) cells to compete with each other, the relative abundance of cDNAs from the cells is determined, e.g., identified and quantified, and then compared to the relative abundance of cDNAs that was present when the cDNAs were first introduced into the cells.

The determination of the specific cDNAs, which become over or under represented in the propagated cells, indicates directly which cDNAs (and proteins) are related to the growth characteristics of the cell. In the case of malignant cells, the under represented cDNAs are the cDNAs (and products) that can be manipulated to perturb growth. In a broader sense, the under represented cDNA are the cDNAs (or genes) which show “synthetic genetic relations” with the circumstances under which the cells are grown, e.g. under environmental stress, the presence of a characteristic mutation, etc. Identification of such relations has been a central concern of many contemporary biologists.

In an aspect of the invention, the cDNA can be identified and quantified, by extracting the total DNA from the cells. All cDNA inserts in the extracted DNA are then copied in a single linear PCR procedure. The copied PCR products can then be converted into tagged species with detectable moieties, such as incorporated biotin or fluorescent nucleotides. The tagged species can be interrogated using commercially available DNA microarrays, originally designed for enumeration of mRNAs.

In a further aspect of the invention, the cDNAs that are used to transfect the cells can be flanked by characteristic sequences, which are not found elsewhere in the genome. Accordingly during identification and quantification of the cDNAs in the propagated cells, the flanked cDNA can be readily copied by PCR. In the PCR process, multiple PCR cycles can be performed using only a downstream primer to yield linear products whose titer is roughly proportional to that of the starting material (unlike standard exponential PCR, which would strongly bias toward the shorter cDNAs). The mixture of single-stranded products is then rendered double-stranded by inclusion of the upstream primer for a single final cycle. The downstream primer can include, for example, sequences corresponding to a promoter that is recognized by T7 RNA polymerase. The double-stranded products can therefore be transcribed with this enzyme to yield products that are employed for probing in microarrays.

### BRIEF DESCRIPTION OF THE DRAWINGS

Further features of the present invention will become apparent to those skilled in the art to which the present invention relates from reading the following description of the invention with reference to the accompanying drawings in which:

FIG. 1 is a schematic view of the cell integrity signaling pathway, flanked by illustrations of the distribution of Mid2-GFP (left) and Wsc1p-GFP (right). Both proteins were expressed from their normal promoters after integration of GFP at the C-terminus. Note that Mid2p is present on much of the cell surface, while Wsc1p is concentrated at the bud surface. B: bud. M: mother cell.

FIG. 2 is a flow diagram illustrating a method in accordance with an aspect of the invention.

FIG. 3 is a schematic illustration of possible outcomes upon growth in galactose, comparing the abundance of individual cDNAs in wsc1-D cells (Wo, W+) to their abundance in isogenic wild type controls (Co, C+). Samples Wo and Co are recovered just before transfer to galactose-containing medium. Samples W+ and C+ are recovered after the cells have been cultured for 900-1800 minutes in galactose medium. We expect the cDNA representations in Wo and Co to be similar to each other and therefore have grouped them together. In reality, C+ will be compared to Co and W+ will be compared to Wo. These two primary comparisons will then be compared to each other. The abundance of each cDNA will also be evaluated in cells grown in glucose medium. These data will be analyzed in parallel with the data for cells grown in galactose. Changes of interest are those seen for wsc1-D in galactose medium (W+ vs Wo) but not in glucose medium (or for wt cells).

FIG. 4 illustrates a covert selection strategy in which each color symbolizes the presence of a distinct ectopic cDNA. Significant changes occur when the cells are grown. cDNAs which sensitize cells to the culture conditions are expected to become depleted, while those which promote growth are expected to become enriched.

FIG. 5 illustrates cDNA quantitation of a typical yeast plasmid for cDNA expression, indicating the flanking targets for PCR amplification. Comparison of transcriptional profiling to SPI. Both procedures generate biotinylated cDNAs using T7 RNA polymerase. In SPI, 30 cycles with the reverse primer are designed to yield linear single-stranded products, which are converted into a double-stranded copy in a single final step upon addition of the forward primer.

FIG. 6 illustrates GC content of subsets of cDNAs in which having determined which of 933 cDNAs are detected on the microarrays, we can divide the group into true positives, false positives, true negatives and false negatives. As shown, each group has nearly the same GC content. False negatives therefore are not due to difficulties in copying sequences of high GC content.

FIG. 7 illustrates a plot showing the mean signal ratio between the groups is approximately a linear function of the relative DNA input.

FIG. 8 illustrates differential cDNA Enrichment and Depletion (Fold Change) Upon Growth. Quadruplicate data sets were used to calculate fold-enrichment for six comparisons. (A) Cells grown at 30° C. in glucose vs the initial sample. (B) Cells grown at 37° C. in glucose vs the initial sample. (C) Fold-changes (C) divided by fold-changes (D). (D) Cells grown at 30° C. in galactose vs 30° C. in glucose. (E) Cells grown at 37° C. in galactose vs 37° C. in glucose. (F) Fold-changes (E) divided by fold-changes (D).

FIG. 9 illustrates (A) six control strains (within the sector) and 26 SPI extreme strains that were streaked on glucose dropout plates (Glc: top) or galactose dropout plates (Gal: bottom) and allowed to grow at 30° C. Strains which show SPI depletion are in the inner circle, while strains which show enrichment are in the outer circle. Growth of all strains is comparable on glucose plates. On galactose plates, the colony sizes of ˜80% of the strains roughly parallels expectations. Examples of controls (C), strains which show increased growth (I) or decreased growth (D) are enlarged at the right. The strains that do not follow expectations could reflect differences between the requirements for growth in liquid vs solid media. Details of two controls (C) and A-G on galactose plates are at the bottom.

FIG. 10 illustrates validation by ds-Linear PCR of triplicate samples of a mixture of three strains grown for ten generations in glucose or galactose dropout medium. Their DNA was used as a template for linear PCR. The largest cDNA (MAL31) shows a SPI enrichment of 54, while the others show <2× change. “0” is the time zero mixed sample before culture. Note the differential retention of the enriched cDNA. Lack of validation may reflect intrinsic differences between growth in liquid and on plates, as well as imperfections in the library which—although widely used—has not been completely sequence verified.

FIG. 11 illustrates plots of cDNAs that show strong differential enrichment in Table II can do so either because of increased growth at 37° C. or decreased growth at 30° C. Correspondingly, cDNAs which show strong differential depletion in Table II can do so either because of decreased growth at 37° C. or increased growth at 30° C. The three panels to the left concern the 100 cDNAs which show the greatest relative depletion at 37° C., while those at the right are the 100 which show the greatest increase. Note, in both cases, the presence of cDNAs which show reciprocal behavior.

FIG. 12 illustrates plots comparing 30° C. SPI Data to Transcriptional Profiles. (A) represents SPI second order fold data that are calculated by dividing the 37° C. vs 30° C. fold change in galactose by the 37° C. vs 30° C. fold change in glucose. In (B-D), the second order SPI data are compared to RNA transcript profiles of the same host cell cultured at 30° C. or 37° C. in glucose or galactose medium. In (B), the RNA signals at 37° C. in glucose are compared to RNA data at 30° C. in glucose. In C, the RNA signals at 37° C. in galactose are compared to 30° C. in galactose. In D, the (37° C. galactose/30° C. galactose) ratio is compared to the /(37° C. glucose/30° C. glucose) ratio. As can readily seen, there is no widespread correspondence between the levels of mRNAs and SPI data. The transcripts which do show strong induction upon addition of galactose include the familiar set of genes GAL1, GAL2, etc.

### DETAILED DESCRIPTION

The present invention relates to a method of identifying complementary DNA (cDNA)(and/or genes) that contribute to cell phenotype, such as cDNA/genes that promote or inhibit cell growth. When applied to yeast cells, the present method makes it possible to identify synthetic relations for greater than about 80% of genes which are “not essential” for haploid growth. The present method also makes it possible to learn how cells cope with the expression of individual mutant proteins. The present method when applied to animal cells further makes it possible to investigate the significance of numerous genes that can be deleted without impairing the development or physiology of animal. Variants of the method in accordance with the invention allow for the identify cDNAs which regulate the ability of cells to survive environmental and genetic stress, to grow in tumor microenvironments, to metastasize, to home, to resist viral infections, etc. The methods of the present invention also provide a novel opportunity to use existing DNA microarray facilities for a purpose other than transcriptional profiling.

In comparison to conventional synthetic lethal screening or high copy suppression, the method of the present invention makes it possible in a single data set to identify multiple candidate cDNAs. Method of the present invention also distinguishes different degrees of importance of cDNAs, judging from based different degrees of cDNA enrichment or depletion and identifies both synthetic survival factors and synthetic lethal factors. Further, in the method of the present invention no subcloning of candidate segments from corrective genomic fragments is required and a minimum of sequencing is required. Moreover, unlike conventional synthetic lethal screening there is no need to retransform candidate mutants with a library, recover colonies, which show altered sectoring, and further analyze corrective plasmids.

The present invention also relates to the identification of genes differentially required for the survival of mammalian cells missing a target gene. The target gene may be, as a non-limiting example, any of a class of genes including tumor suppressor genes and mutator genes, the function of which is absent or reduced in cancer cells. This invention relates to use of synthetic lethal screening to identify genes that are more important to the growth or survival of cells missing a particular target gene as compared to how important those genes are for the growth or survival of wild-type cells with the target gene. This information is used to rationalize a drug target in mammalian cells of defined genotype. Given a cell with an inactivating mutation in its version or homolog of the target gene, a synthetic lethal screen might identify genes X, Y and Z in that cell, each of which have a more deleterious phenotype as double mutants with the target gene mutants than as single mutants. Upon identification of gene products X, Y and Z (or their homologs) in mammals, these gene products would be rationalized as drug targets for the elimination of cells missing the target gene.

In accordance with the method of the present invention, a plurality of cells are provided. The plurality of cells can include, for example, yeast cells or eukaryotic cells (e.g., animal cells). The yeast cells can be, for example, wild type yeast cells or mutated yeast cells, such as yeast cells that do not synthesize the transmembrane protein Wsc1p. The animal cell can be, for example, normal animal cells or abnormal cells, such as tumor cells (e.g., murine melanoma cells) or neoplastic cells. By way of example, the plurality of cells can be malignant cells that are harvested from a mammal with a malignancy (e.g., cancer).

Each cell of the plurality of cells is then effected to express a single ectopic cDNA. The ectopic cDNA expressed by each cell can differ from the ectopic cDNA expressed by other cells. The cells can be effected to express the single ectopic cDNAs by, for example, transfecting (or infecting) the cells with an ectopic cDNA library vector at a low multiplicity. The low multiplicity is such that each cell can be transfected with a single vector that includes the ectopic cDNA.

A “vector” (sometimes referred to as gene delivery or gene transfer “vehicle”) refers to a macromolecule or complex of molecules comprising cDNA to be delivered to a target cell, either in vitro or in vivo. Vectors include, for example, viral vectors (such as retroviruses), plasmids, liposomes and other lipid-containing complexes, and other macromolecular complexes capable of mediating delivery of the cDNA to a target cell.

Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector cDNA by the cell; components that influence localization of the polynucleotide within the cell after uptake (such as agents mediating nuclear localization); and components that influence expression of the cDNA. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors, which have components or functionalities mediating binding and uptake), or vectors can be modified to provide such functionalities.

One example of a viral vector that can be used is a retrovirus. The structure and life cycle of retroviruses makes them ideally suited for introducing cDNA into the cells since (i) the majority of sequences coding for their structural genes are deleted and replaced by the cDNA of interest which are transcribed under control of the retroviral regulatory sequences within its long terminal repeat (LTR) region and (ii) they replicate through a DNA intermediate that integrates into the host genome.

In addition to viral vector-based methods, non-viral methods may also be used to introduce a cDNA into a target cell. A review of non-viral methods of DNA delivery is provided in Nishikawa and Huang, Human Gene Ther. 12:861-870, 2001. One example of a non-viral cDNA delivery method according to the invention employs plasmid DNA to introduce a cDNA nucleic acid into a cell. Plasmid-based gene delivery methods are generally known in the art. Synthetic gene transfer molecules can be designed to form multimolecular aggregates with plasmid DNA. These aggregates can be designed to bind to the target cells. Methods that involve both viral and non-viral based components can also be used according to the invention.

The cDNA library that provides the cDNA, which is introduced into the cells, can comprise cDNAs with differing nucleic acid sequences and lengths. These nucleic sequences can code for genes. By way of example, the cDNA library can comprise a collection of cDNA clones that were generated in vitro from mRNA sequences isolated from an organism or a specific tissue or cell type or population of an organism. In order to expand the repertoire of cDNAs, the cDNA library can also be subject to random mutagenesis before use.

The cells effected with the cDNA library can be propagated (or grown) for a growth interval in an environment that allows the cells to compete against one another. This environment can be, for example, a cell culture or an animal (e.g., immunodeficient mouse). The cells can be grown under conditions and for sufficient time to allow for at least 5, at least 10, at least 15, 20, about 20, at least 20, at least 30, at least 40, at least 50, 60, about 60, or at least 60 population doublings. This competitive outgrowth period allows for the amplification of any differential growth rates of cells due to the expressed cDNA. For example, if a cell in a population of cells grows at a rate only 5% slower than the other cells of the population, that cell will be depleted from the population by 50% as compared to the other cells of the population after 20 population doublings. A control experiment can also be performed in which cells not effected with the cDNA are grown under the same conditions for the same amount of time.

After the growth interval, which allows the infected (or transfected) cells to compete with each other, the relative abundance of cDNAs from the cells is determined, e.g., identified and quantified, and then compared to the relative abundance of cDNAs that was present when the cDNAs were first introduced into the cells. Deviations from the initial distribution are functionally important, e.g. promote or inhibit cell survival/growth. The specific cDNAs that become over or under represented in the propagated cells indicates directly which cDNAs (and proteins) are related to the growth characteristics of the cell. In the case of malignant cells, the under represented cDNAs are the cDNAs (and products) that can be manipulated to perturb growth. In a broader sense, the under represented cDNA are the cDNAs (or genes) which show “synthetic genetic relations” with the circumstances under which the cells are grown, e.g. under environmental stress, the presence of a characteristic mutation, etc. Identification of such relations has been a central concern of many contemporary biologists.

The identified cDNAs can become targets for therapeutic intervention. For example, cells from a patient could be subject to these procedures, involving culture either in the laboratory, or maintenance as xenografts in immunodeficient mice. The resulting analysis would allow tailoring of therapy to the individual and would not require former knowledge of the molecular lesions, which are present.

In an aspect of the invention, the cDNA can be identified and quantified, by extracting the total DNA from the cells. All cDNA inserts in the extracted DNA are then copied in a single linear PCR procedure. The copied PCR products can be converted into tagged species with detectable moieties, such as incorporated biotin or fluorescent nucleotides. The tagged species can then be interrogated using commercially available DNA microarrays, originally designed for enumerations of mRNAs.

The use of a DNA microarray to detect fluorescent or biotinylated tags has been published (Winzeler, E. A., et al., 1999, 285:901-906, incorporated by reference in its entirety). Such arrays can be produced by one of skill in the art according to established protocols (Marton, M. J., et al., 1998, Nat Med 4(11):1293-301) or obtained commercially (Affymetrix Inc., Santa Clara, Calif.). Each address of the DNA microarray contains DNA complementary to a known DNA tag. After hybridization, the amount of fluorescence detectable at a given location in the DNA microarray reveals the relative abundance of the DNA bearing the tag complementary to the DNA at that location. The software programs can be used for analysis of these arrays and are well suited for data processing, statistical evaluation, visual display etc.

In a further aspect of the invention, the cDNAs that are used to transfect the cells can be flanked by marker sequences, which are not found elsewhere in the genome. Accordingly, during identification and quantification of the cDNAs in the propagated cells, the flanked cDNA can be readily copied by PCR. In the PCR process, multiple PCR cycles are performed using only a downstream primer to yield linear products whose titer is roughly proportional to that of the starting material (unlike standard exponential PCR, which would strongly bias toward the shorter cDNAs). The mixture of single-stranded products is then rendered double-stranded by inclusion of the upstream primer for a single final cycle. The downstream primer can include, for example, sequences corresponding to a promoter that is recognized by T7 RNA polymerase. The structure of these products allows them to be converted into tagged species that include either biotin or fluorescent moieties (e.g., Cy3, Cy5 and fluorescein). These species can then be interrogated commercially available DNA microarrays, originally designed for enumeration of mRNAs.

### EXAMPLES

### Example 1

Many phenotypically “silent” mutations (e.g. deletion of gene X, or the presence of a particular mutant protein) are compatible with survival since cells synthesize appropriate compensatory proteins and avoid the expression of others. By identifying these functionally related proteins, one can characterize the biological significance of Xp, and therefore develop indirect means of regulating those function in which it participates.

Differential plasmid representation (DPR) will first be used to compare wild type yeast to an isogenic mutant, which grows well but does not synthesize the transmembrane protein, Wsc1p. Classical genetic and biochemical approaches have already characterized Wsc1p, which is thought to function as a “stress sensor” and initiate signaling to the “cell integrity” pathway. Both wt and mutant strains are transformed with a centromeric cDNA library. The mixture of transformants is then grown in selective medium under conditions, which induce expression of a single ectopic cDNA in each cell. This causes competition among the many transformants, which represent a diverse set of genetic backgrounds. After 10-20 generations, DNA is recovered from both types of transformant and determined, using DNA microarrays, the relative abundance of each of the plasmids which was originally present. Each plasmid should retain its initial relative abundance if it provides no growth advantage or disadvantage. Comparison of the enrichment/depletion data for wt vs mutant strains therefore makes quantitative predictions as to which cDNAs exhibit a positive or negative “synthetic” relation with deletion of Wsc1p.

DPR is substantially simpler than classical approaches for identification of “synthetic lethal” interactions between genes (in which a second gene must continue to be transcribed if a first gene is mutated or deleted). It also makes it possible to detect interactions, which are less accessible by classical methods. In these “synthetic survival” interactions, mutation of one gene is compatible with survival only if a second gene is not expressed.

The PKC or “Cell Integrity” Signaling Pathway

To establish DPR, a strain that carries a deletion of the “stress sensor,” Wsc1p is studied. The following section describes the biology of Wsc1p and the “PKC” MAP kinase pathway, which it regulates. As explained in Preliminary Data, our study of the Arrest of Secretion Response (ASR) has caused us to investigate the function Wsc1p and Wsc2p. The choice of Wsc1p for the studies therefore extends our previous work, as well as serving as prototype for development of DPR.

Distinct MAP kinase pathways in yeast are implicated in many events. The “PKC” path, which is named after the only protein kinase C in S. cerevisiae, is important for cell cycle progression, polarization of growth and of the cytoskeleton, cell wall synthesis, and the responses to changes of tonicity, to heat shock and to mating pheromone. Since mutation of Pkc1p causes cell lysis unless osmotic support is provided, this pathway is also referred to as the “cell integrity” pathway. Pkc1p can trigger the corresponding downstream MAP kinase cascade, and also acts along an “alternative” effector path.

The PKC MAP kinase pathway activates a critical transcription factor, Rlm1p, which in turn can activate genes implicated in cell wall synthesis (Fks2p etc.), as well as Swi4p/Swi6p. Pkc1p receives input from the cell surface via the GTPase, Rho1p, which is activated when PI4,5P2 binds its guanine nucleotide exchange factor, Rom2p. Interestingly, Rho1p also functions with Fks1p in the 1,3-β-glucan synthase complex.

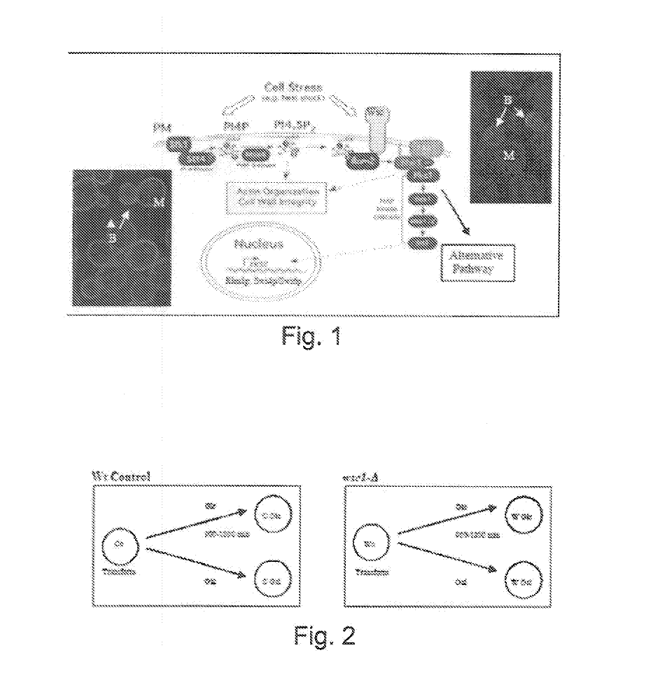

Rho1p signaling also requires Wsc1p (cell wall integrity and stress response component, also referred to as Slg1p and Hcs77p), a single-pass transmembrane glycoprotein of the surface of the bud (FIG. 1). Wsc1p is non-essential under standard growth conditions, but is required for thermotolerance and resistance to several other varieties of stress (H₂O₂, ethanol, agents causing DNA damage). Wsc1p was first detected in a screen to identify mutants which require a hyperactive MEKK along the Pkc1 MAP kinase path, as a suppressor of a swi4 mutation, and as a suppressor of the heat shock sensitivity of strains that overexpress RAS-GTP. Some of these functional characteristics as well as sequence comparisons demonstrate the close relation between Wsc1p and Wsc2p, Wsc3p, Wsc4p and Mid2p. The latter proteins have been studied much less than Wsc1p. As Wsc Alternative Pathway Rlm1p, Swi4p/Swi6p shown in FIG. 1, at least Mid2p has a distinct distribution. Other stress sensors must also function in yeast, considering that transcriptional stimulation upon heat shock does not require Wsc proteins, e.g. stimulation mediated by heat shock response elements, stress response elements and AP-1-like response elements.

Overview of Research

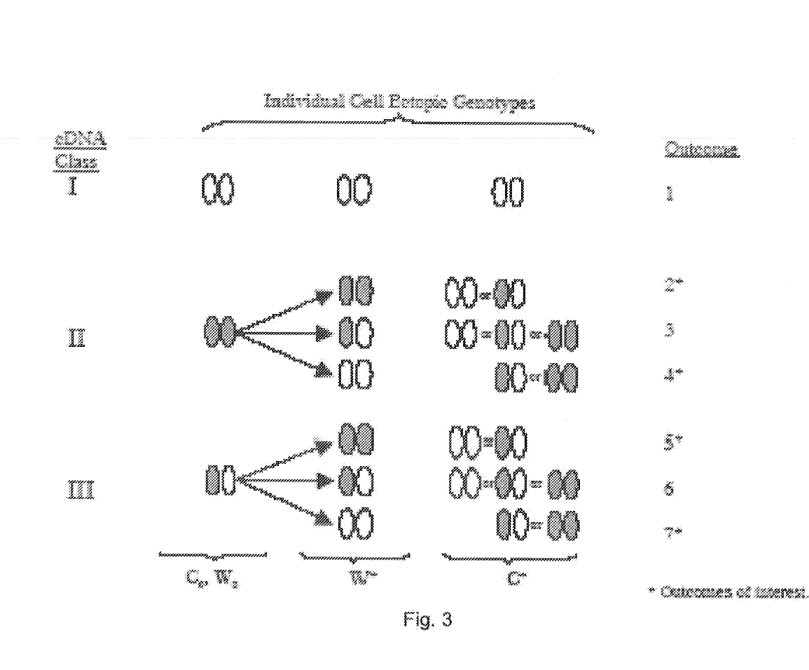

wsc1-Δ grows well under standard laboratory conditions. Considering that even quadruple Wsc mutants are viable, its survival may be due to a variety of other proteins. A genetic selection should be able to identify at least some of these proteins. We will proceed as shown in FIG. 2, with all cultures being performed in triplicate:
A galactose-inducible yeast cDNA library is used to transform a wt (C: control) and an isogenic wsc1-Δ strain (W). Both are kept in raffinose selective medium and samples Co and Wo are washed and set-aside after 2 generations (t=0).
Both transformants are then cultured for 10-20 generations in selective medium with galactose (or glucose) to allow any inequality of growth rate to occur. If a given cDNA confers no advantage or disadvantage, the relative abundance of the corresponding transformant should not change. If it inhibits growth, it will become underrepresented. If it promotes growth, it will become overrepresented.
DNA microarrays are used to determine the relative abundance of the ectopic cDNAs in wsc1-Δ, by comparison to wt—after correction for changes which occur in glucose medium.
The data is validated by deliberately overexpressing differentially represented cDNAs and testing their impact, and by monitoring corresponding mRNA levels.
A conventional synthetic lethal screen starting with wsc1-Δ is conducted in order to learn whether it identifies a subset of the synthetic relations which are identified by DPR.

There are several distinctive features of DPR, by comparison to conventional synthetic lethal screening or high copy suppression:
DPR makes it possible—in a single data set—to identify multiple candidate cDNAs.
It should be able to distinguish different degrees of importance of cDNAs, judging from different degrees of plasmid enrichment or depletion.
DPR identifies both synthetic survival factors and synthetic lethal factors.
No subcloning of candidate segments from corrective genomic fragments is required.
A minimum of sequencing is required. Moreover, unlike conventional synthetic lethal screening, there is no need to retransform candidate mutants with a library, recover colonies, which show altered sectoring, and further analyze corrective plasmids.
DPR does not require preliminary construction of plasmids which carry wild type copies of the genes being investigated (e.g. with color markers such as ADE3) and the use of host strains with corresponding mutations which make colony sectoring visible.

The readout from experiments resembles that of transcriptional profiles of cells, since both approaches use DNA microarrays. Nevertheless, transcriptional profiles describe the levels of multiple transcripts, which are expressed in the same cell, and do not justify any conclusions as to which specific changes are responsible for growth or survival of the mutant. By contrast, the present approach will identify single supernumerary cDNAs, which are over- or underrepresented in cells, and their mere presence should (with some exceptions) imply that they affect growth. It is true that the expression of one “extra” cDNA can modify expression of many genes; however, in this case a single identifiable “master” cDNA will be responsible in each transformant.

Preliminary Data

We have previously studied the impact on yeast of heat stress, osmotic stress, and arrest of the secretory path. We have extensive experience in molecular cloning and the use of genetic selections, e.g. as part of studies of temperature-sensitive mutants, which do not export mRNA from the nucleus at the restrictive temperature. We also have worked extensively with animal cell mutants which are deficient in the biosynthesis of GPI anchored proteins.

The Arrest of Secretion Response (ASR) in Yeast—“Signaling from Within”

The secretory path can be interrupted at multiple levels using distinct temperature sensitive sec mutants. Our DNA microarray analysis shows that many transcripts encoding cell surface permeases are strongly induced in sec mutants. Components of the Hog1 MAP kinase signaling pathway—which often opposes the effects of the PKC pathway—are also stimulated. Work from the Warner lab (and our microarray data) also shows that these mutants down regulate transcription of mRNAs encoding ribosomal proteins. Moreover, our studies show that sec mutants relocate a number of nucleolar/nuclear proteins to the cytoplasm. We collectively refer to these changes as the “Arrest of Secretion Response” (ASR) and expect that they are adaptive since they cause the cell to stop expending metabolic energy for ribosome synthesis. The ASR is distinct from the unfolded protein response (UPR); the ASR can occur in a strain, which does not allow the UPR (ire1-Δ). Moreover, sec mutants do not upregulate the folding equipment of the ER or protein implicated in “ER-associated degradation.”

Our experiments show that both Wsc1p and Wsc2p are required for the ASR and that their activity in the ASR requires their being trapped along the secretory path. They thus can report on the functionality of the secretory path. The ASR also requires protein kinase C (Pkc1p), but not the PKC MAP kinase cascade. Consistent with this latter observation, the transcriptional consequences of stimulation of this cascade (by overexpressing Mkk1p) are distinct from those of the ASR. The Warner laboratory has reported that down regulation of ribosome synthesis by tunicamycin involves Wsc1p and Pkc1p.

The ASR is likely to be closely related to many aspects of cell physiology. For example, the ASR provides a novel means to adjust cell growth. Moreover, the presence of a single mutant protein, which cannot leave the ER, can inhibit transport of other proteins along the secretory path. Such “bystander” effects could cause Wsc-like proteins to accumulate along the secretory path. Such indirect effects may well elicit ASR-like signaling, regulate growth, and contribute to the incidence of some sporadic diseases.

Lack of Importance of Rap1p

Rap1p binding sites are found upstream of most ribosomal protein transcriptional units. We therefore have performed two types of experiments to learn whether Rap1p biology is affected upon interruption of the secretory path in sec1-1. Neither indicates that Rap1p is involved.
We have constructed a sec1-1 strain, which expresses functional GFP-tagged Rap1p. The tagged protein clusters at telomeres and is also diffusely distributed in the nucleus. This distribution does not change upon transfer of the cells to 37° C. for 2 hr.
Rap1p is required for silencing of mating type information HML and HMR and rap1 temperature-sensitive strains therefore show a “bimating” phenotype upon incubation at the restrictive temperature. We have shifted either rap1 ts strains or sec1-1 to 32° C. or to 37° C. for up to 5 hr and then challenged them with tester strains of both mating types to evaluate their ability to mate (at 23° C.). Although rap1-2 and rap1-5 do mate with either tester, sec1-1 mates only with the opposite mating type.
   An ASR in Higher Eukaryotes

We observed that at least one nuclear protein, hnRNP A1, relocates to the cytoplasm of HeLa cells upon interruption of the secretory path for several hours with brefeldin A. As for yeast, relocation is abrogated by simultaneous inhibition of protein synthesis. A functional equivalent of Wsc may therefore exist in higher eukaryotes.

Molecular Studies of Wsc2p

To investigate how Wsc proteins initiate signaling, it is important to know which portion of their structure is of functional importance. We therefore have performed random mutagenesis of a plasmid expressing Wsc2p from a galactose inducible promoter. We have generated several dozen mutants which are toxic when expressed in wt cells grown on galactose medium, but not on glucose medium. If the toxicity of these plasmids requires Pkc1p, we will sequence them. We expect that some such dominant negative mutants have altered the structure of the cytosolic tail of Wsc2p and therefore may be useful for identification of downstream signaling intermediates.

Experimental Design

After optimization of transformation procedures, both the wt strain and an available isogenic wsc1-Δ mutant are transformed with a yeast library in which transcription of cDNAs is driven by a GAL1 promoter. We avoid genomic libraries since:
Most plasmids from genomic libraries carry more than one gene.
Each gene would be expressed at a level dictated by its own promoter.
Selection against genes whose expression inhibits growth is impossible, since the corresponding transformants vanishes immediately upon transformation.

We have previously used galactose-inducible cDNA libraries, which were constructed in a pRS316-GAL1 vector. Since the number of copies of individual yeast mRNAs ranges from 0.3 to over 200 per cell, such libraries are highly biased. We therefore, instead, use a “defined” cDNA library, which includes approximately equimolar amounts of plasmids corresponding to a large majority of yeast ORFs. There are several possible choices: copper- or galactose-inducible libraries for expression of ORFs as GST fusions and a library based on Gateway vectors. For simplicity, we assume that a galactose-inducible library is used.

Transformation in non-inducing medium is conducted so as to give 3-5× coverage of the genome, i.e. 20,000-30,000 transformants. Judging from replica plating experiments with the pRS316 library mentioned above, 15 out of a total of 25,000 transformants did not grow on galactose. This low incidence of lethality, coupled with our intention to use more modest levels of induction, make it unlikely that these considerations will seriously limit DPR. Possible positive or negative effects on growth due to the presence of any truncated cDNAs should also not be a problem if Affymetrix microarrays are used, since these arrays include oligos from both the beginning and ends of transcripts, making it possible to discard any data which show length bias.

Pooled wt transformants and (separately) wsc1-Δ transformants are cultured at 30° C. in selective raffinose medium. Samples are then removed for DNA purification at t=0 (2 generations) and 900-1800 minutes after transfer to 2% glucose of 1% galactose/1% raffinose (i.e. about 10-20 generations, as in FIG. 2). The OD600 of the cultures is maintained between 0.1 and 1.0, which will cause any differences in cell concentrations to increase exponentially.

Insert DNA sequences from all samples are copied by PCR, using primers which lie just lateral to the multicloning site. The downstream primer includes sequences corresponding to a T7 RNA polymerase promoter so that in a final step biotinylated cDNAs are generated, as in standard Affymetrix protocols for transcriptional profiling. It will be appreciated that other DNA methodologies could be used for the proposed studies (e.g. Agilent arrays which are interrogated using pairs of red/green labeled probes); however, the excellent facility at this University presently uses Affymetrix arrays (see letter).

In Affymetrix transcriptional profiling, one proceeds as follows:
1) Harvest total RNA from a cell population,
2) Purify poly(A)+ RNA from the total RNA,
3) Generate a complementary DNA strand, by priming reverse transcriptase (Superscript II) with an oligo-dT primer which includes downstream sequences corresponding to the promoter of T7 RNA polymerase,
4) Generate a double-stranded cDNA using E. coli and T4 DNA polymerases,
5) Generate biotinylated cDNA from this duplex using T7 polymerase, biotinylated CTP and UTP,
6) Fragment the cDNA to a length of 35-200 bases,
7) Use the biotinylated cDNA to probe Affymetrix microarrays,
8) Wash and then add streptavidin-phycoerythrin (which can be followed by biotinylated anti-streptavidin and a second layer of streptavidin-phycoerythrin),
9) Quantitate fluorescence on each spot, subtract background, calculate ratios of intensity.

After copying the cDNA inserts and adding T7 polymerase promoter sequences to them, the equivalent of steps #5-9 are followed. These steps are performed at the DNA Microarray Facility by the same individuals who are responsible for transcriptional profiling.

The biotinylated cDNA products are then used at the University DNA Microarray facility to interrogate Affymetrix yeast S98 DNA microarrays. The data sets are analyzed with Affymetrix Micro DataBase software and Data Mining Tools, as well as with SpotFire, GeneSpring and Significance Analysis of Microarrays (SAM), which are routinely in use for transcriptional profiling. These allow statistical analysis of significance, hierarchical clustering, etc. We have previously used this facility and these arrays. All cultures are performed and analyzed in triplicate.

We can thus learn whether there is significant enrichment or loss of selected cDNAs in the wsc1-Δ (galactose culture 900-1800 min vs t=0), by comparison to the wt (galactose culture 900-1800 min vs t=0). Among those cDNAs, which are over or underrepresented, we are also able to calculate a hierarchy of enrichment (or depletion) which will be important for prioritizing follow-up experiments.

Outcomes

The period of continued culture in the presence of glucose will surely exert unspecified selective pressures on the initial cell populations, and therefore affect the differential enrichment of cDNAs, e.g., due to the varied size of the inserts in the plasmids. These data will provide a baseline for the data on cells grown in galactose medium, where larger changes are expected. Transcripts whose titer is significantly increased due to plasmid transcription are likely to cause the greatest increment in levels of individual proteins and therefore—in general—have the largest effects.

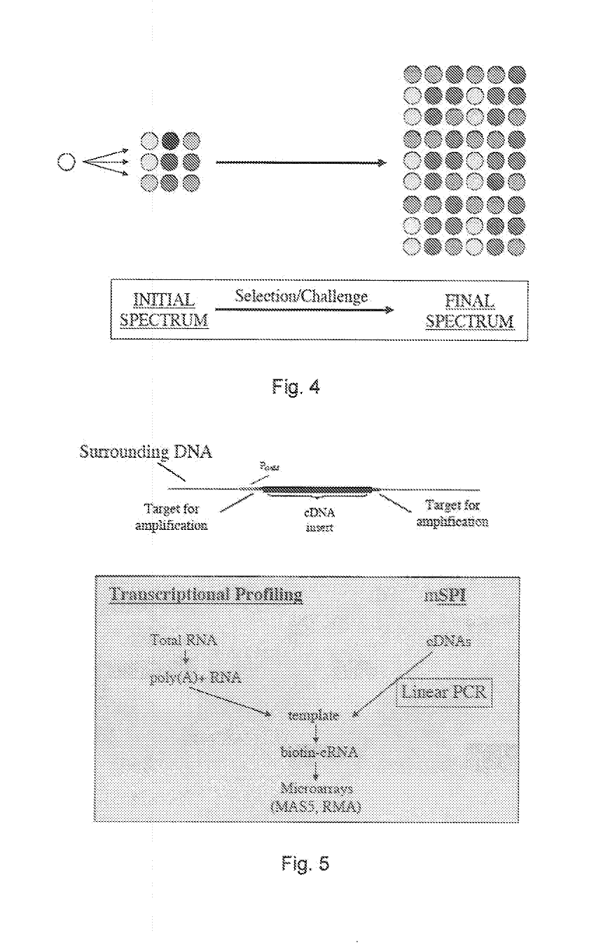

FIG. 3 illustrates the fundamental concept behind the data analysis of cells grown in galactose-containing medium. cDNAs of interest (class II and III) are those whose representation in wsc1-Δ is either much more or much less than in wt.

Class I cDNAs

Due to a transformation efficiency <100%, some cDNAs could be absent.

Class II cDNAs

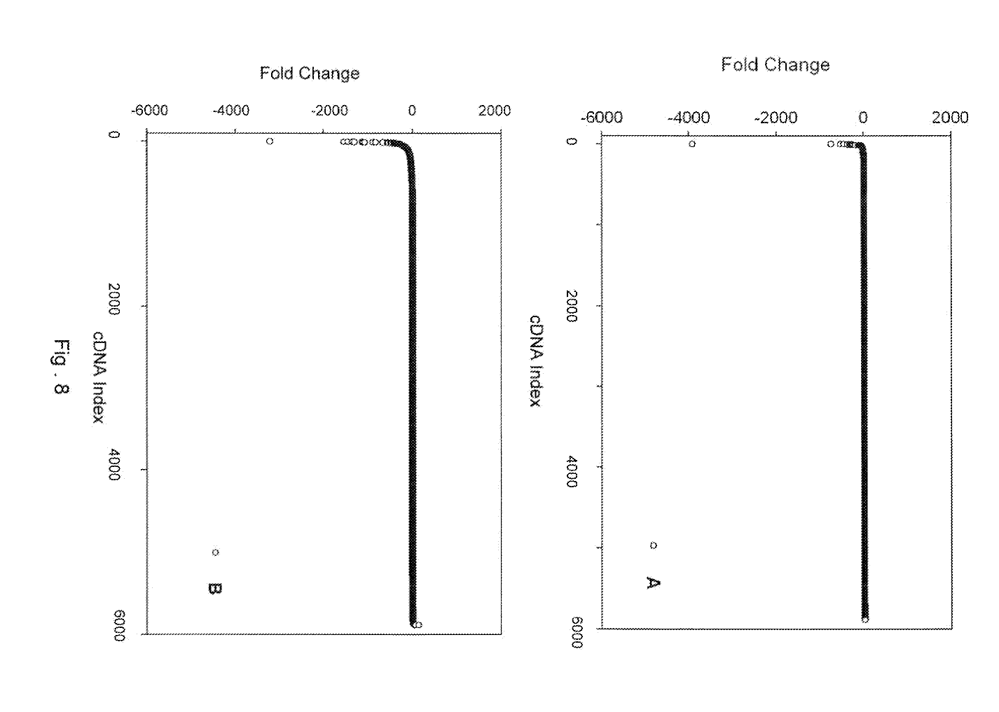

These cDNAs will be highly represented in the W₀ and C₀ samples. This is indicated in the FIG. 5 by having each of the two cell symbols darkened. After growth for 2000 minutes with galactose (middle column), there are three possibilities for wsc1-Δ (W⁺): these cDNAs could persist in all cells, in some cells, or in no cells. Which outcome is of interest depends on whether the same cDNA persists in sample C⁺. If a given cDNA is enriched in W⁺, but is poorly represented in C⁺ (outcome 2), it could favor survival in the absence of Wsc1p. If a given cDNA is underrepresented in wsc1-Δ, but is present in sample C⁺ (outcome 4), it could be selectively incompatible with the lack of Wsc1p.

Class III cDNAs

These cDNAs will be represented at intermediate levels in sample C₀ and W₀. This is indicated by having only one of the two cell symbols darkened. In wsc1-Δ after 2000 minutes, there are again three possibilities: these cDNAs could persist in all cells, in some cells, or in no cells. Which outcome is of interest again depends on whether the same cDNA persists in sample C⁺. If a given cDNA is enriched in W⁺, but is poorly represented in C⁺ (outcome 5) it could favor growth in the absence of Wsc1p. If a given cDNA is very underrepresented in wsc1-Δ, but is present at intermediate or high levels in sample C⁺ (outcome 7), it could be selectively incompatible with the lack of Wsc1p.
In pilot experiments, we will compare the spectrum of cDNAs which is present in wsc1-Δ after culture in media with 0.1 to 1.0% galactose (+1.9% to 1% raffinose). We expect that maximal induction will produce the most extreme differences of cDNA representation, but it is not clear to what extent the initial conditions (1% galactose) are already exaggerated. If these pilot experiments allow us to define less extreme conditions (e.g. 0.25% galactose, 1.75% raffinose) which cause changes in cDNA abundance which are comparable to those seen with 1% galactose, we will modify the growth conditions, accordingly. In parallel, we will vary the duration of culture. Prolonged culture will emphasize the impact of relatively minor differences in growth rate and therefore increase sensitivity; however, it may also increase the noise level—which would be evident by comparing triplicate cultures.
We expect to detect overrepresentation of cDNAs encoding proteins which strengthen the cell wall or are functionally related to Wsc1p, e.g. Wsc2p-Wsc4p, Mid2p, Rom2p, and downstream components along the Pkc1p signaling pathways. We may observe under representation of cDNAs encoding proteins, which weaken the cell wall, e.g., proteins, which promote endocytosis or degradation of cell wall components.
   Limitations

DPR is appropriate for detecting cDNAs, which encode proteins related to the functions of Wsc1p. Nevertheless—as in other genetic selections—there is no reason to expect that DPR will detect all cDNAs of interest. Limiting considerations areas follows:
Mutagenesis: Transformation can be mutagenic and therefore affect growth rates.
Silencing: Some cDNAs (and cells) could persist if their expression is silenced.
Level of Expression: Certain cDNAs which encode proteins which promote survival/growth of wsc1-Δ could go undetected because the increment in expression from the plasmid does not significantly boost protein levels, or because excessive protein levels are inhibitory.
Mathematical Considerations: If some cDNAs are overrepresented (or underrepresented) for biological reasons there will inevitably be others that are underrepresented (or overrepresented) for mathematical reasons.
   Data Validation
   mRNA Levels

If the over- or under representation of a given cDNA is physiologically meaningful, we expect that it will approximately parallel transcript levels. For example, if a cDNA is absent, but the corresponding mRNA is abundant, it is not possible to sustain the hypothesis that the cDNA is inhibitory. Moreover, if a given cDNA is relatively abundant, the corresponding mRNA must be detectable. For this reason, RNA recovered from each of the cell population will be analyzed by Affymetrix transcriptional profiling. These data will reflect the composite expression from chromosomal loci and from the ectopic cDNAs.

Small Scale Repeats

A subgroup of the most over- and most underrepresented cDNAs (10 altogether) will be retested, using fresh samples of both cells and plasmid, on both wt and wsc1-Δ, to learn whether they affect growth rate. We predict that those cDNAs which were overrepresented in wsc1-Δ grown on galactose will promote growth of wsc1-Δ, while those which were low or absent will diminish growth, both by comparison to their effects on wild type cells. Inhibition of the expression of some cDNAs, which were over represented, should also inhibit growth.

Future Applications of DPR

Our first emphasis is on Wsc1p because it is important for the “cell integrity” pathway and for the ASR. One could also use DPR to investigate the functional significance of over expression of individual proteins. Equivalent experiments could identify cDNAs, which affect growth upon exposure to specific drugs or forms of environmental stress.

The largest prospect is actually for higher eukaryotic cells, since there is little opportunity to work with banks of deletion cell lines. One would use retroviral libraries to integrate single cDNAs into the genome (e.g., using a wt and corresponding mutant host cells) to study cell survival after deletion of non-essential genes (e.g., the tumor suppressor, p53); to learn how cells and organisms can survive for many years despite the presence of proteins which are ultimately toxic, such as polyglutamine-expanded forms of Huntington which are characteristic of Huntingdon's Disease; to identify genes which govern the sensitivity of cells to specific drugs or various forms of environmental stress, etc.

The research described above, rather than starting with candidates whose significance is investigated individually, will identify the group of cDNAs (a “molecular signature”) whose presence correlates positively or negatively with growth of wsc1-Δ. Since there is little reason to expect deviations from the initial representation of cDNAs unless they provide selective advantage (or disadvantage), it is very likely that they are responsible for differences of growth rate. Once such cDNAs have been identified, one can work from the top down—i.e., knowing that they are functionally important. Moreover, there will at once be an indication of which are the most important. By focusing on an already well-characterized deletion strain (wsc1-Δ), we anticipate being able to add to understanding of the “cell integrity” pathway and the ASR. Identification of novel genes, which exhibit synthetic relations with WSC1, will motivate investigations of their fundamental biology. Related methodology will become appropriate for parallel investigation of higher eukaryotic cells.

When applied to yeast, DPR should make it possible to identify synthetic relations for the >80% of genes which are “not essential” for haploid growth. It should also make it possible to learn how cells cope with the expression of individual mutant proteins. When applied to animal cells, DPR should make it possible to investigate the significance of numerous genes, which it turns out can be deleted without impairing the development or physiology of mice. Further variants of the procedure should make it possible to identify cDNAs which regulate the ability of cells to survive environmental and genetic stress, to grow in tumor microenvironments, to metastasize, to home, to resist viral infections, etc. DPR should also provide a novel opportunity to use existing DNA microarray facilities for a purpose other than transcriptional profiling.

### Example 2

### Covert Genetic Selections to Optimize Complex Phenotypes: Growth Regulators for S. cerevisiae

We developed Single Promoting or Inhibiting (SPI) to identify cDNAs, which impact complex phenotypes. This procedure is based on cDNA enrichment (“covert selection”) rather than overt correction of cellular phenotype. It therefore accesses relations, which are inaccessible via classical genetic selections. We grow a pool of S. cerevisiae transformants, which carry single plasmids from a cDNA library and the initial cDNAs and those, which persist at 30° C. are quantitated on microarrays. These data make specific predictions as to which cDNAs affect growth, emphasizing the importance of translation initiation, ribosome biogenesis and ER glycosylation. In equivalent experiments at 37° C., the positive impact of both GPI-anchored proteins and mitochondrial function becomes evident. SPI thus provides a streamlined genome-wide selection for cDNAs which can affect cell performance. SPI provides functional insight under circumstances in which classical genetic approaches cannot be implemented. Identification of such genes is critical for orienting therapeutic interventions.

Introduction

Cell growth, migration, and ability to resist stress and infection depend on many factors which have been the subject of focused investigations. Further elucidation of these determinants is of intrinsic interest and also can lead to the design of corresponding molecular therapies. Nevertheless, when multiple components are involved, it often is unclear which one(s) should be targeted in order best to affect cellular responses.

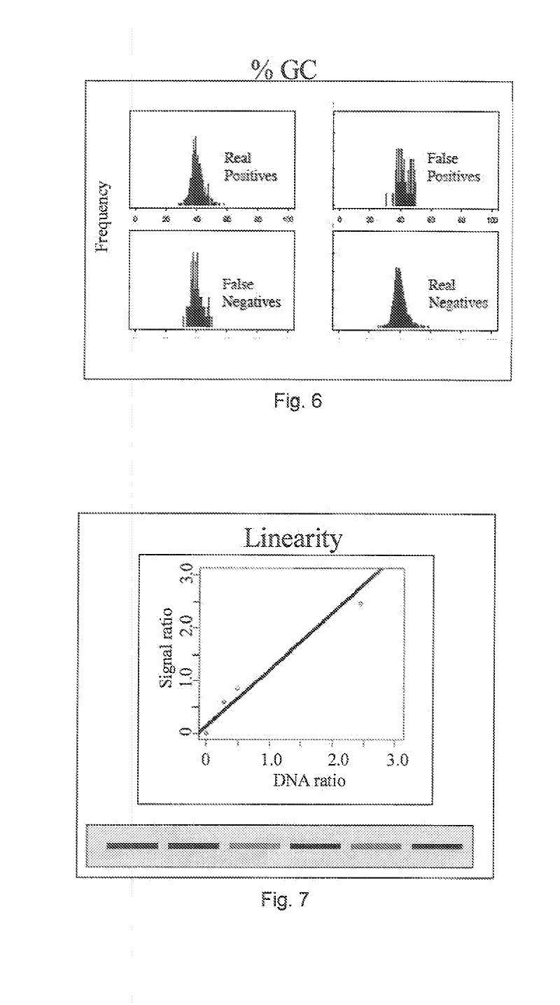

The search procedure, which is described in this below, identifies single cDNAs from high complexity libraries whose overexpression or depletion can have the greatest impact on complex phenotypes. To exemplify this approach, we have established a pool of strains of S. cerevisiae which differ from each other with regard to single ectopic cDNAs, and have then determined which of these cDNAs/cells becomes more or less abundant upon growth in liquid culture (FIG. 4). The central premise is that a cDNA, which has no impact on cell growth/survival will cause its host—and that cDNA—to remain at an intermediate level of abundance. CDNAs that promote growth or survival will cause the corresponding cells and their cDNAs to become more abundant, while those that impair growth, will have inverse effects. The selection for cDNAs of interest is therefore “covert,” in that the readout does not depend on any gross alteration of phenotype, e.g. growth rate. Moreover, since there is no need to begin by testing each cDNA individually, these experiments do not require panels of deletion strains or transformants.

Result

The pool of strains carries a “defined” synthetic library of 5885 plasmids in which each cDNA is under control of a galactose-inducible promoter. The availability of these constructs makes it possible to generate an approximately uniform mixture of corresponding transformants without induction, and then to test the impact of their expression when galactose is added. To develop procedures for plasmid recovery, copying of cDNA inserts, and probing of microarrays, we have first worked with small subsets of transformants and shown that the abundance of each plasmid is approximately uniform in pooled DNA extracts.

The cDNA inserts are all present in a constant context in the library plasmids. To copy them without introducing the length and abundance bias which is characteristic of conventional PCR, we developed a linear PCR procedure (“ds-Linear PCR”) which in a final step generates double-stranded products. Biotinylated cDNAs transcribed from them are then used to interrogate DNA microarrays under the same conditions as for transcriptional profiling (FIG. 5).

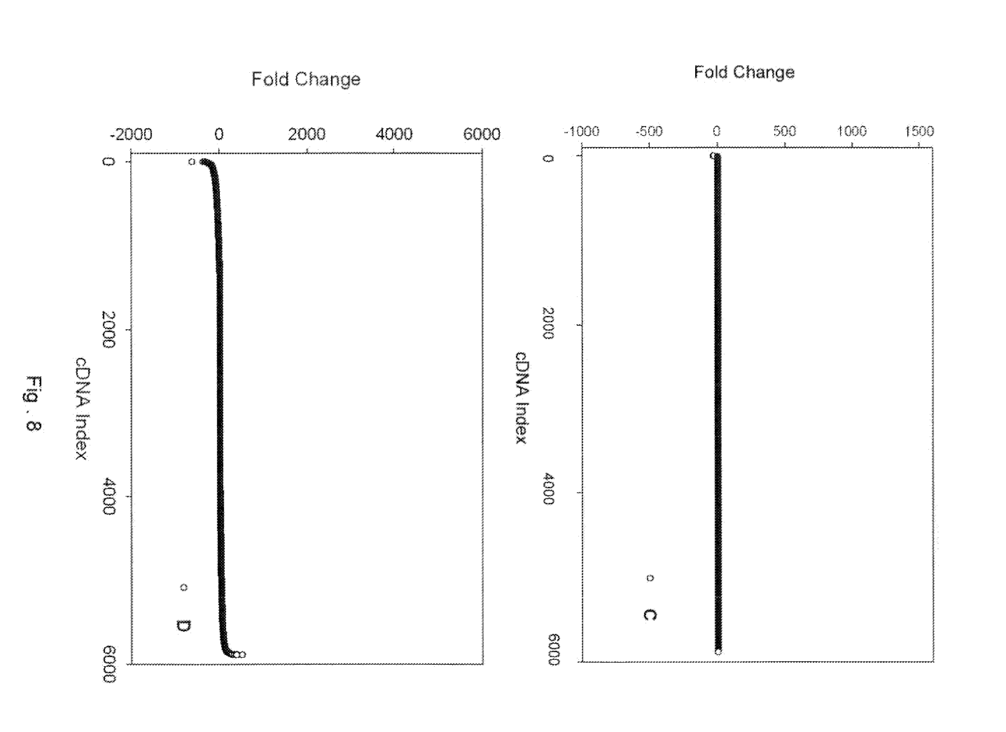

By processing a pooled subset of 933 transformants, we are able to tabulate the number of loci for which signals are both expected and are detected (true positives), loci for which signals are expected but are not detected (false negatives), loci for which signals are not expected but are detected (false positives), and loci for which signals are not expected and are not detected (true negatives). In this situation, there are 94% real positives and 6% false negatives. The false negatives are not enriched in cDNAs of high CG content, as might have been expected if they were difficult to copy (FIG. 6).

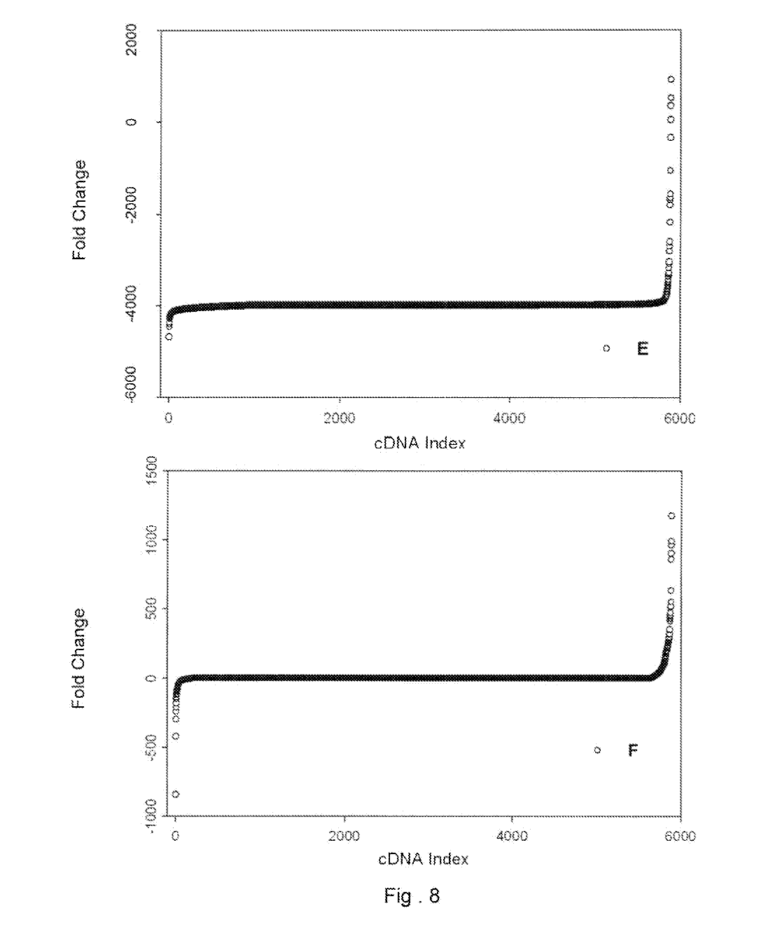

When DNA is recovered from two distinct pools of about 1000 transformants and different proportions are mixed and processed, the ratio of the mean signal values for each pool is nearly proportional to its relative abundance over the range examined (FIG. 7). In biological experiments, a two-fold change in relative signal intensity therefore corresponds to a comparable change in cDNA input.

Analysis of Yeast Growth

We have inquired whether the pool of 5885 transformants retains its initial cDNA spectrum (i.e., the fluorescent intensities for all cDNAs) in liquid culture in selective medium under several conditions. Those which change to the greatest extent, the tails of the distributions, are referred to as “SPI Extremes.”

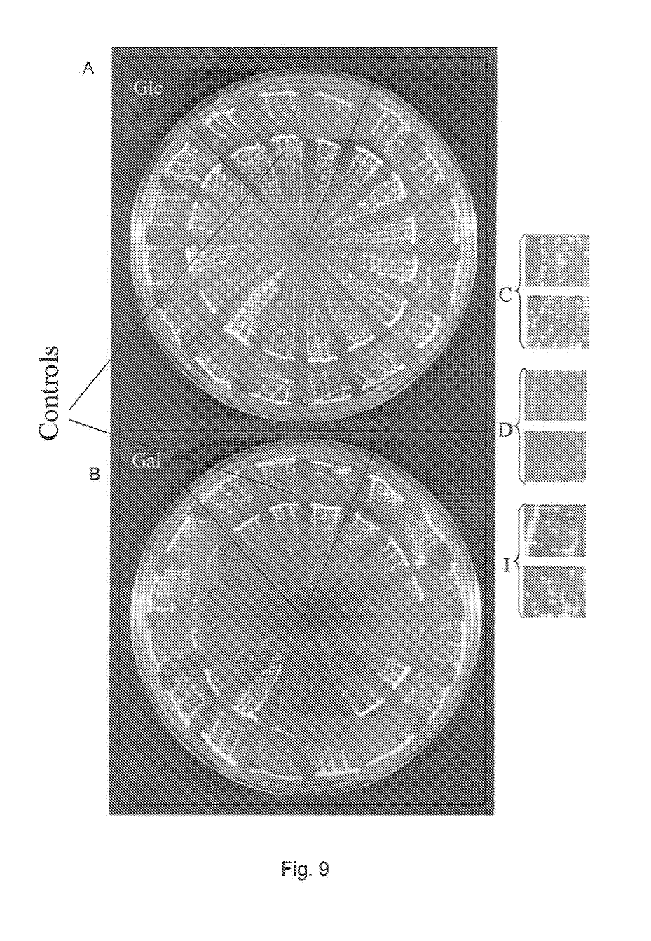

When quadruplicate cultures are maintained for 30 generations at 30° C. without induction (i.e., in glucose medium), there is little change of relative abundance of most cDNAs; however, SPI Extremes can already be identified, with negative fold changes of several hundred-fold being obvious, while increases are very modest (FIG. 8A). cDNA depletion may result from sequestration of unidentified critical factors by the DNA inserts which are present. FIGS. 8B and 8C show that the spectrum, which persists at 37° C. in glucose medium, is nearly identical to that with persists at 30° C. in glucose medium.

To identify cDNAs which affect growth upon deliberate overexpression, we have cultured quadruplicate samples of the same mixture of transformants in galactose medium for 30 generations at 30° C. and compared their cDNA spectra to that of cells cultured in glucose medium (FIG. 8D). Small numbers of cDNAs show distinct several hundred-fold enrichment or depletion. Considering that the library which we have used results in significant production of most of the proteins encoded by cDNAs, it is notable that yeast tolerates at least some overexpression of a large fraction of its genome.

Since transformants can be cultured in galactose medium at temperatures up to 37° C. without loss of viability, parallel experiments have also been conducted at this modestly stressful temperature (FIG. 8E). Under these conditions, there are more examples of strong enrichment, while decreases remain modest.

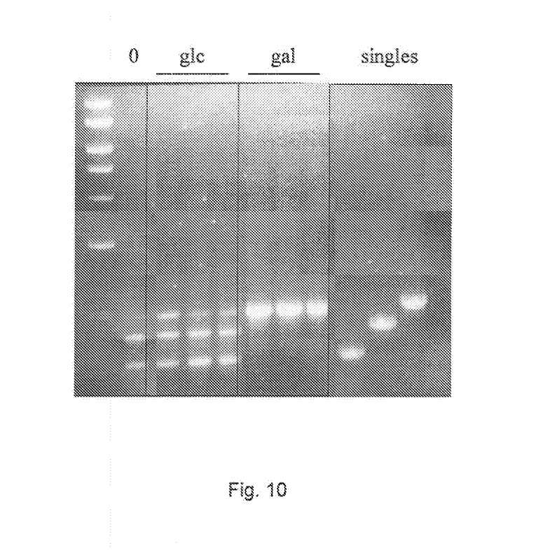

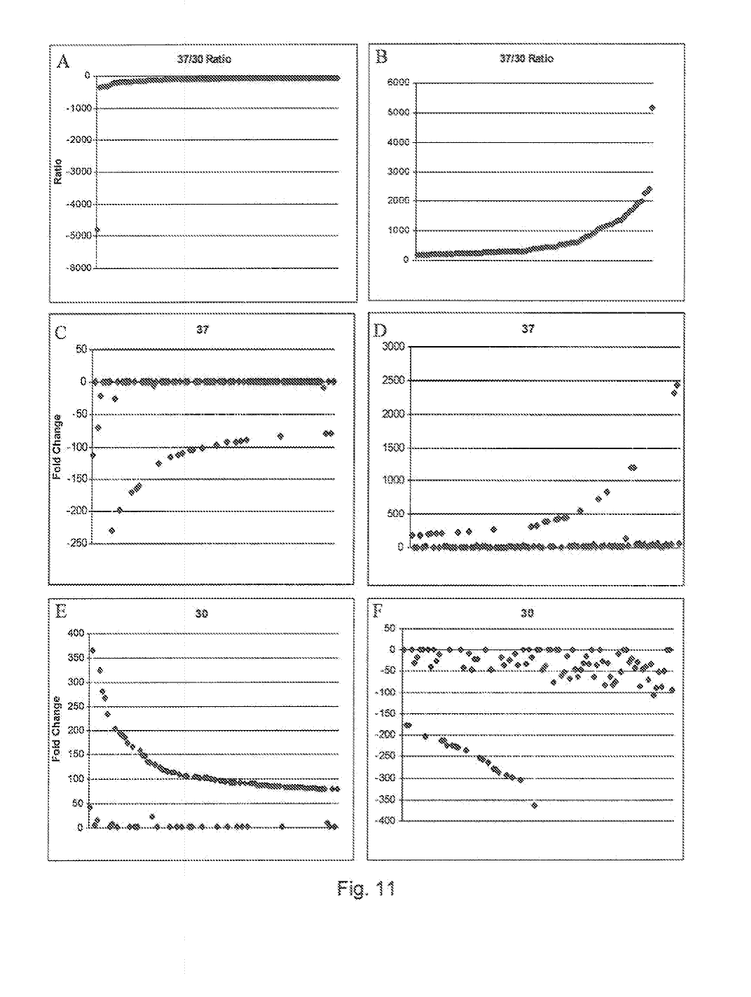

By comparing data sets for cells cultured in galactose medium vs glucose medium, we can minimize the possible contribution to SPI Extremes of host cell mutations which affect growth. To further control for their possible impact, we have turned to three validation tests for SPI Extreme cDNAs (FIGS. 9-10). About 80% of cDNAs that are predicted to have a strong positive or negative impact can be confirmed as being stimulatory or inhibitory upon examination of their growth on galactose vs glucose plates. Moreover, several have been confirmed by monitoring the growth of small pools of transformants in liquid medium and then copying their cDNAs by ds-Linear PCR, or by subcloning inserts into a vector which allows their induction from a distinct promoter and assessing growth on plates.

cDNAs of Interest

To our knowledge, there has been no previous indication that the rate of growth of yeast at either 30° C. or 37° C. can be increased by overexpression of single proteins. Seventeen cDNAs among the 100 most enriched cDNAs at 30° C. are also among the 100 most enriched at 37° C. (Table I).

TABLE I
cDNAs which are among the top or bottom 100 at both 30° C.
and 37° C.

| | | FC | FC |
|---|---|---|---|
| Name | Function/Comment | 30° C. | 37° C. |
| AKR2 | Palmitoyl transferase ? | 502 | 4045 |
| BNA1 | Nicotinic acid biosynthesis | 158 | 141 |
| BRF1 | Pol 1 transcription factor | 287 | 100 |
| EGD2 | NAC complex | 192 | 100 |
| FUN12 | Translation initiation | 210 | 137 |
| GLS2 | ER glucosidase | 274 | 653 |
| LSM3 | mRNA decay | 159 | 157 |
| LSM8 | mRNA decay | 157 | 92 |
| MF(ALPHA)1 | Mating pheromone | 111 | 519 |
| RPL5 | Large ribosomal subunit assembly | 175 | 4522 |
| RPS9A/B | Translational fidelity | 243 | 7457 |
| SLM1 | Actin stress response | 161 | 325 |
| SSU1 | Sulfite efflux | 155 | 923 |
| SVS1 | Vanadate resistance | 167 | 2361 |
| THI80 | Thiaminepyrophosphate- | 144 | 4919 |
| | phosphokinase | | |
| UTP10 | U3 snoRNA complex | 363 | 4345 |
| YSP1 | mitochondrial | 144 | 650 |
| ADY4 | SPB during meiosis | −164 | −140 |
| AKR1 | Palmitoyl transferase | −250 | −113 |
| ATR1 | Multidrug pump | −141 | −115 |
| CDC47 | MCM complex | −161 | −108 |
| GEM1 | Outer mitochondrial membrane | −184 | −115 |
| | GTPase | | |
| HDA1 | HDAC complex | −150 | −113 |
| NCA2 | Expression of F₀-F₁ ATPase | −617 | −224 |
| NUP49 | Nucleoporin | −149 | −103 |
| PBS2 | MAPKK for hypertonic stress | −217 | −149 |
| PCT1 | Phosphatidylcholine synthesis | −147 | −119 |
| POG1 | Transcriptional activator/pheromone | −153 | −259 |
| SCS3 | Inositol phospholipid synthesis | −317 | −457 |
| SPT8 | Histone acetylase complex (SAGA) | −226 | −113 |
| TRM1 | tRNA methyl transferase | −182 | −104 |
| | (nucleus/mitochondria) | | |

Six encode proteins that are critical for protein synthesis or ribosome genesis and therefore have the interesting property that they affect the titer or transport of many other proteins. These are:
Fun12p/eIF5B, a GTPase which functions in translation initiation by promoting binding of Met-tRNAᵢ^{Met} to small ribosomal subunits.
Egd2p, the α subunit of the NAC complex which binds nascent polypeptide chains and is thought to influence their delivery to the ER.
Rps9A/B, a conserved small ribosomal subunit protein which is a major determinant of translational fidelity.
Utp10p, a protein associated with U3 snoRNA which is required for 18S rRNA synthesis.
Rpl5p/L1, which is required for assembly of large ribosomal subunits.
Brf1p, a subunit of the RNA polymerase III transcription factor, TFIIIB).

Additional enriched cDNAs which are within the top 100 at both temperatures and encode proteins which are plausibly needed for optimal growth are:
Gls2p/Rot2p, one of the subunits of the endoplasmic reticulum glucosidase II. This enzyme trims the N-glycans of newly-synthesized glycoproteins after their folding in the ER and prior to exit to the Golgi. It is also required for efficient ER-associated degradation. Suboptimal maturation and expression of one or more of its glycoprotein substrates—or perhaps a cell wall component such as 1,6-β-glucan—could normally limit cell growth.
Lsm3p and Lsm8p, which function in mRNA decay.
Slm1p, which regulates actin organization in response to stress.

Moreover, given that the host cell is MAT a, it is striking that the cDNA encoding the alpha mating pheromone is enriched.

There is no reason to expect functional coherence among the inhibitory cDNAs since interference with many functions can be detrimental. Table I lists those which are most depleted at both temperatures. It is curious to find the cDNA encoding palmitoyl transferase, Akr1p, since it is closely related to the putative palmitoyl transferase, Akr2p, which is highly enriched.

SPI can identify cDNAs which protect cells against stress or sensitize them to stress. For this purpose, we have evaluated the fold-enrichment of cDNAs at 37° C. vs 30° C. in galactose (FIG. 8F). It is notable that several groups of cDNAs are among the 100 which show the highest differential enrichment (Table II), including 18 which encode mitochondrial proteins and 12 which contribute to expression of GPI-anchored proteins, several of which are implicated in stress resistance. Other groups of differentially enriched cDNAs encode proteins which are required for DNA synthesis or mRNA splicing factors, as well as a few which are required for vacuole function or biogenesis, for tRNA maturation or peroxisome biogenesis.

TABLE II
Differential Enrichment 37° C. vs 30° C.
Top 100 for 37° C./30° C.

| | | DNA | mRNA |
|---|---|---|---|
| Mitochondrial | GPI Anchoring | Synthesis | Splicing |
| ADH3, CEM1, | CSH1, GAS2, GAS5, GPI1, | ADE13, | HSH155, |
| COQ6, COX10, | GPI10, GPI14, GPI18, LCB5, | ELG1, | PRP4, |
| COX11, HEM4, | PER1, TSC10, UTR2, YPS1 | RAD27, | PRP42 |
| HMI1, IDP1, | | RNR2 | |
| MBA1, MDM1, | | | |
| MDS1, MIA40, | | | |
| MMM1, | | | |
| MRPL11, | | | |
| MSR1, NAM2, | | | |
| OSM1, POR2 | | | |

Among the cDNAs which are most depleted at 37° C. by comparison to 30° C. in galactose medium are nine which are related to actin function or cell polarity, five related to microautophagy, and five related to chromatin remodeling.

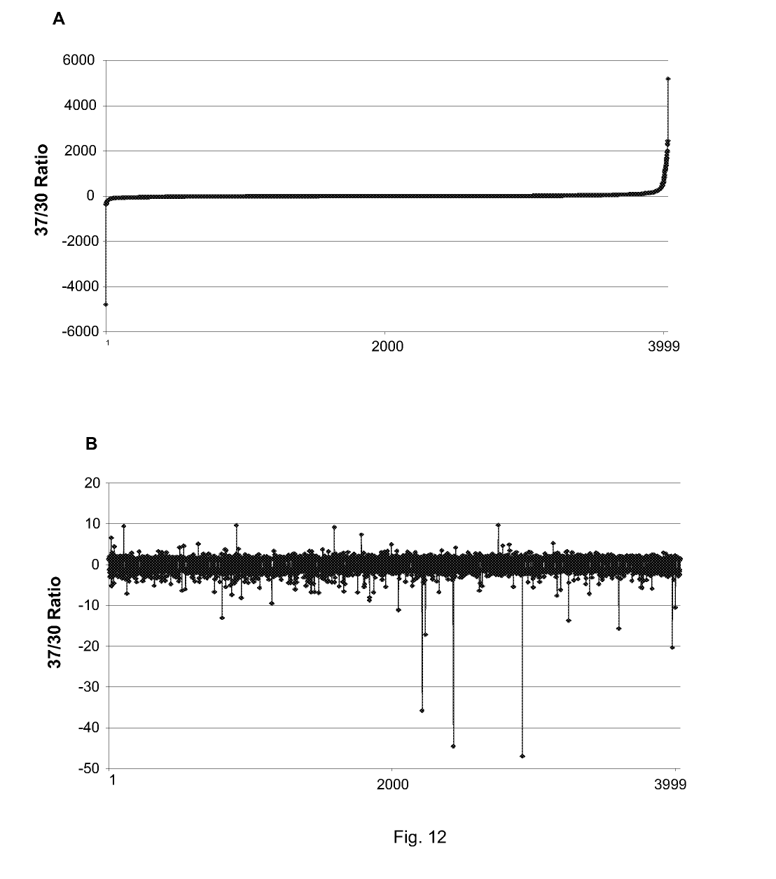

Such calculations are clearly a composite, including cDNAs which are highly enriched at 37° C. as well as those which are especially depleted at 30° C. FIG. 11 compares the 37° C. vs 30° C. data to both the enrichment at 37° C. and the depletion at 30° C. This analysis will make it possible to identify the limited subset of cDNAs whose overexpression (or depletion) is most likely to be useful for promoting growth at 37° C.

Discussion

Classical gene complementation strategies can be used to investigate diseases of monogenic origin but are seldom applied to diseases of more complex origin. The present study shows that such strategies can be extended to the analysis of the impact of high temperature, providing an example of a phenotype of complex multifactorial origin. A fundamental difference between SPI and classical complementation cloning is that the goal is to identify a spectrum of “contributory genes” (or cDNAs), rather than a single entity (“command gene”), and that these contributory genes have graded impact on cellular phenotypes. Some of those cDNAs which confer a seemingly minor growth advantage (or disadvantage) can become dramatically enriched (or depleted) as a function of growth. A second fundamental difference is a loosening of the concept of genetic selection, since in SPI the selection is evident at the level of differential cDNA enrichment, rather than overt phenotypic correction. The two attendant characteristics of SPI which are integrally part of this strategy are the expression of single ectopic cDNAs, and the inclusive readout which is afforded by microarrays.

The realization that multiple cDNAs can promote cell growth may seem at variance with the concept of there being a single rate-limiting-step. Nevertheless, such multiplicity is characteristic of optimization strategies for complex systems, reflecting the intricate interactions among components which contribute to the sustenance of the whole. Clearly, the optimization strategy of SPI could be made more stringent by prolonging the growth/selection interval. Moreover, it could be iterated to identify dependent groups of functionally significant genes, e.g., by starting with a cell in which on SPI extreme cDNA is already overexpressed (or depleted).

Several of the positively-acting cDNAs which we have identified pertain to events which have previously been recognized as important control points: ribosome synthesis and translation initiation, for example, are generally considered to limit the speed of cell growth and are implicated in oncogenesis. Moreover, censorship of glycoprotein exit from the ER is certainly central to expansion of the cell wall. No candidates are obviously related to maintenance of plasmid number.

Previous laborious studies have used panels of S. cerevisiae transformants to evaluate the ability of individual cDNAs, GST fusions and gene fragments to inhibit growth. These qualitative assays evaluate clonal growth on plates, while SPI is based on a competition assay in liquid culture and lends itself to quantitative comparisons. It is of particular interest that the one study which has used the same library that we have used is the study which agrees most closely with the present work. It identified 1702 inhibitory cDNAs, while for SPI there are 1274. 341 are shared by both.

One might expect that the SPI Extreme (enriched) cDNAs would correspond to essential genes; however, as for the total genome, only about 20% of the cDNAs which are most enriched at 30° C. (or 37° C.) correspond to genes which are essential under standard growth conditions (Saccharomyces Genome Database). Neither FUN12 nor GLS2/ROT2 for example, is essential. Thus, survival of the organism under laboratory conditions cannot require those genes which have the potential to be most beneficial. These “accessory” beneficial genes represent an evolutionary opportunity, which can be detected by SPI.

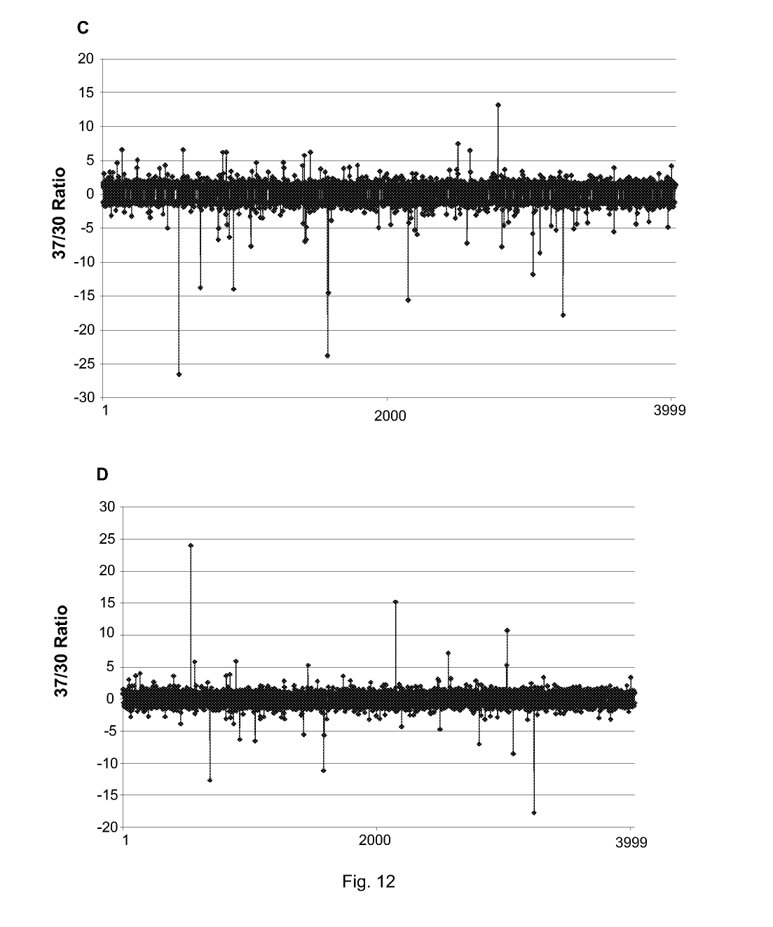

It is also of interest to ask whether SPI extreme enriched cDNAs correspond to mRNAs which are upregulated at 37° C. (in either glucose or galactose medium). As shown in FIG. 12, there is minimal concordance. This discrepancy may signify that the normal circuitry of gene expression seldom allows the cell to manipulate the level of single transcripts, i.e. bystander transcripts which would be co-induced sabotage any attempt to upregulate those, which by themselves, could be most useful.

SPI is well-suited to identify cDNAs whose up- or downregulation can protect against environmental stress or genetic stress in yeast. We feel that the greatest prospect for implementation of SPI will be in the context of human biology, where it should again exemplify the utility of genetic approaches outside the normal realm of genetic inquiry. This is because of the difficulty of devising reasoned therapeutic strategies for diseases of complex origin, because of the need to understand the asymptomatic gross phenotype of many gene knock-outs, to learn how to control cell migration, to promote resistance to infectious organisms, etc. Indeed, in each of these situations, it is likely that subtle selective events are always at work. Unlike transcriptional profiling, the information content of SPI is very high since it makes specific predictions as to which single cDNA is functionally important.

Experimental Procedures

Cells and cDNA Library

These materials were obtained from M. Snyder and D. Gelperin (Yale University). The haploid host cell was YC123=SF657-2D=Snyder strain 258 (MATa pep4-3 his4-580 ura3-52 leu2-3, 112). The 10 kb pEG(KG) 2μ vector used for cDNA expression carried both URA3 and leu2-d selectable markers and appends GST to the N-terminus of each product (Mitchell et al., 1993). Frozen stocks of single transformants and pools of transformants were prepared by standard methods.

Cell Growth, Plasmid and RNA Recovery

Liquid cultures were established from single colonies. After growth in uracil dropout medium at room temperature, aliquots were diluted to A600=0.1 using uracil dropout glycerol-lactate medium (2% glycerol, 2% lactate, 0.05% glucose, 0.67% bacto-yeast nitrogen base without amino acids, pH5.5), grown overnight at room temperature to A600=1-2 and then harvested by sedimentation.

5 ml samples of cells were washed with water, broken by vortexing with glass beads, and extracted with a Qiagen DNA extraction kit.

To evaluate cell viability, samples were stained with FUN1 (Molecular Probes (F-7030)) and examined by epifluorescence. Living cells showed bar-shaped orange structures in the vacuole while dead cells lacked this signal and were predominantly green.

To study growth at 30° C. and 37° C., frozen pools including all 5885 strains were thawed, washed, and then adjusted to A600=0.05-0.1 in glycerol-lactate medium. 5 ml at OD600=1-2 was set aside and refrozen to provide a t₀ sample. Duplicate cultures were supplemented with 2% glucose or 2% galactose and then shaken at 30 and 37° C. Growth was monitored at 600 nm and aliquots of each culture were rediluted to A600=0.05-0.1 so that the A600 never exceeded 1.5. Duplicate 5 ml cultures were snap frozen in liquid nitrogen after a total of 30 generations.

For RNA analysis, we used hot phenol to extract logarithmic cultures growing in glycerol-lactate medium supplemented with 2% glucose- or 2% galactose and processed the samples in accordance with Affymetrix protocols.

Ds-Linear PCR Amplification

Linear amplification of cDNA inserts was performed using a mixture of DNA polymerases, starting with thirty reverse cycles using a primer which includes the T7 phage RNA polymerase sequences and concluding with a single forward cycle. The printers were complementary to sequences which flank the inserts and do not include the regions which encode GST or the GAL promoter.

Microarray Analysis

Ds-linear PCR products were pooled, concentrated and purified using a Qiagen column. In general, 200 ng of PCR product was used to generate biotinylated cRNA probes for S98 DNA microarrays. Samples were processed at the University Affymetrix facility, scanning the microarrays with a GeneArray scanner and preprocessing the data using GeneChip Operating Sortware. In all experiments described below we studied independent quadruplicates and based analysis on those cDNAs for which signals are classified as “Present” in each sample, using the MAS5 algorithm. Normalized signals from totally independent replicates have a mean correlation coefficient of 0.97.

To learn whether cDNAs which give only a weak signal at t=0 can be studied reproducibly, we asked whether there is any correlation between initial signal intensity and the consistency of their presence (or absence) after 30 generations. We do not detect any such correlation using a students t-test.

Data from quadruplicate samples were used to get a weighted mean signal for each probe using MAS5, and these data were then imported into GeneSpring GX 7.3.1 software (Silicon Genetics, Redwood, Calif.) and normalized. To identify cDNAs which show statistically significant differences when comparing samples derived from cells cultured under different conditions, we used a Welsh t-test and calculated the Benjamini and Hochberg False Discover Rate. We retained those entries with fold changes of >2 and p<0.05 in all 16 comparisons so long as, for increases m the denominator entries were considered “present” or, for decreases, the numerator values were considered “present.” The averages of the 16 calculated fold-changes were then calculated and used to produce the enrichment or “S-plots” and to identify the most enriched and most depleted cDNAs. Fractional values reflect depletion and are represented according to the following convention: 0.1=−10; 0.2=−5 etc.

Validation

Growth of individual transformants were studied by streaking single colonies onto solid media (2% glycerol, 2% lactate in uracil drop-out medium, pH5.5+2% glucose or +0.5% raffinose and 2% galactose). Alternatively, mixtures of cells were cultured in liquid media identical to those used for the initial experiment, processed using ds-Linear PCR and the resulting double-stranded DNA products were then resolved on Agarose gels.

To monitor growth in the absence of galactose induction, selected cDNAs (without the GST moiety) were copied by conventional PCR and subcloned into a URA3 vector in which transcription was under control of a MET25, methionine-repressible, promoter.

### Example 3

### An in vivo Genetic Selection to Prioritize cDNAs Which Regulate Cell Growth

The microenvironment within tumors is often characterized by restricted nutrition and poor oxygenation, the presence of host defense factors, etc. Moreover, survival in this environment requires specific reciprocal interactions with adjacent stromal cells. Logical strategies for elimination of tumors thus could rely on sensitizing malignant cells to such environments. Such sensitization could result from over expression of proteins, which limit growth, or from reduced expression of proteins, which are critical for survival. It is therefore important to identify genes whose under- or over expression—although not responsible for transformation per se—favors or opposes cell growth within the tumor microenvironment.

We have developed a powerful search procedure to identify cDNAs, which contribute to complex phenotypes. This method has already made it possible to identify cDNAs, which govern cell growth upon exposure to environmental stress.

The procedure includes the following steps: 1) growing tumor cells in culture, 2) infecting them with a retroviral cDNA library at low multiplicity, 3) allowing the cells to continue to grow for many generations, either in culture or in immunodeficient mice, 4) using a modified version of the polymerase chain reaction to copy the cDNA inserts present in the cells, 5) identifying and quantitating the cDNAs by using the PCR product to interrogate DNA microarrays. Attention is then focused on those cDNAs whose abundance changes the most, by comparison to their abundance at the moment of infection, and by comparison to parallel experiments with normal cells.

cDNAs are expected to retain their initial relative abundance unless they confer some selective advantage or disadvantage upon the host cell. Therefore—to combat tumor cell growth—those cDNAs which have become most abundant should be down regulated, while those which have become most depleted should be up regulated.

In an aspect of this method, murine melanoma cells will be infected with a retroviral murine cDNA library at low multiplicity so that each cell will express a single ectopic cDNA, with expression being driven by the viral LTRs. The mixture of cells will then be used to generate subcutaneous tumors in immunodeficient mice. The spectrum of cDNAs, which persists, will then be identified and quantitated using a combination of linear PCR and microarray methods. The essence of the procedure is the postulate that—as the cell population grows—the relative abundance of cells expressing each cDNA reflects its impact on cell survival and division.

Since cDNAs which promote or restrict cell growth outside of the tumor microenvironment are not of primary interest, measurements made in the tumor microenvironment will be compared to experiments conducted with the same cells in culture. Ectopic cDNAs, which are differentially overrepresented in the tumors, should be down regulated in an attempt to combat growth. Ectopic cDNAs which are differentially underrepresented in the tumors should be studied to determine whether their over expression will reduce tumor cell growth in vivo. When novel genes are identified which fit these criteria, they will thus become prime targets for further investigations and for therapeutic intervention.

By contrast, to transcriptional profiling of normal vs tumor cells, the present approach selects individual cDNAs which are of functional importance. This approach makes no assumptions with regard to the underlying molecular genetic lesions, which are present.

We have chosen melanomas because well-characterized cell lines are available and because malignant melanoma is one of the fastest-growing malignancies in man. In studies extending beyond those for which support is requested, this approach will be applied to identify cDNAs which regulate the growth of metastases, affect cell sensitivity to radiation and chemotherapeutic agents, etc.

In the broadest view, this method will make possible cancer therapy, which is tailored, to individual patients. For this purpose, malignant cells from the patient will be processed essentially as for the melanoma. This will make it possible to learn which cDNAs are the most potent regulators of growth and survival of these cells.

Having described the invention the following is claimed:

1. A method of identifying complementary DNA (cDNA) that contribute to cell phenotype, the method comprising:
providing a plurality cells;
effecting the cells so that each cell expresses a single ectopic cDNA;
propagating the cells for a growth interval in an environment that allows the cells to compete against one another; and
determining the relative abundance of cDNAs from the propagated cells

2. The method of claim 1 further comprising comparing to the relative abundance of cDNAs from the propagated cells with the relative abundance of cDNAs present when the cells were effected prior to propagation.

3. The method of claim 1, the cells being effecting by transfecting the cells with a cDNA library provided in vectors.

4. The method of claim 3, the cells being transformed at a multiplicity such that each cells is transfected with a single vector.

5. The method of claim 1, the relative abundance of the cDNAs in the propagated cells being determined by identifying and quantifying the cDNA.

6. The method of claim 1, the relative abundance of cDNAs in the propagated cells being determined by copying the cDNA in the propagated cells using a downstream primer to yield linear products, forming double stranded cDNA products from the linear products with an upstream printer.

7. The method of claim 6, further comprising attaching a detectable moiety to the doubled stranded cDNA and interrogated the cDNA with a micro-array.
